(19) United States
(12) Patent Application Publication
(10) Pub. No.: US 2008/0194528 A1
(43) Pub. Date: Aug. 14, 2008

(54) Pharmaceutical compositions comprising organopolysiloxane elastomers and solubilized bioactive compounds

(75) Inventors: Nathalie Barthez, Nice (FR); Emilie Tonglet, Golfe Juan (FR); Sandrine Orsoni, Mandelieu (FR); Laurent Fredon, Roquefort Les Pins (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH AND DEVELOPMENT, BIOT (FR)

(21) Appl. No.: 12/000,773

(22) Filed: Dec. 17, 2007

Related U.S. Application Data
(63) Continuation of application No. PCT/FR2006/001357, filed on Jun. 15, 2006.

(30) Foreign Application Priority Data
Jun. 17, 2005 (FR) 0506183

(51) Int. Cl. A61K 31/59; A61P 17/06

(52) U.S. Cl. 514/167

(57) ABSTRACT

Stable, anhydrous pharmaceutical compositions contain an organopolysiloxane elastomer and, as bioactive ingredient, at least one vitamin D compound in a solubilized form, and are useful for the topical treatment of psoriasis and other skin disorders/afflictions.

### CROSS-REFERENCE TO PRIORITY/PCT APPLICATIONS

This application claims priority under 35 U.S.C. § 119 of FR 05/06183, filed Jun. 17, 2005, and is a continuation of PCT/FR 2006/001357, filed Jun. 15, 2006 and designating the United States (published in the French language on Dec. 21, 2006 as WO 2006/134272 A1; the title and abstract were also published in English), each hereby expressly incorporated by reference in its entirety and each assigned to the assignee hereof.

### BACKGROUND OF THE INVENTION

1. Technical Field of the Invention

The present invention relates to the formulation of bioactive agents for the purpose of topical pharmaceutical applications.

The present invention relates more particularly to stable, anhydrous pharmaceutical compositions comprising an organopolysiloxane elastomer and, as active ingredient, at least one vitamin D derivative in a solubilized form, to the process for preparing same and to its use for the topical treatment of psoriasis and other skin disorders/afflictions.

2. Description of Background and/or Related and/or Prior Art

Vitamin D and derivatives thereof are generally administered in dermatology in the treatment of psoriasis because they limit the excessive production of skin cells on affected surfaces and possess known advantages for the treatment of this condition which is characterized in particular by the presence of thick, squamous, dry lesions.

It is known that a certain number of active ingredients which have an advantageous therapeutic activity are sensitive to oxidation and undergo in particular chemical degradation which results in a substantial loss of their activity in the presence of water. Vitamin D or certain of the vitamin D derivatives are in particular unstable in aqueous media, especially in an acidic environment; they exhibit maximum stability at pH values of around 8.

Consequently, it is advisable to formulate these active ingredients in anhydrous-type compositions.

The anhydrous compositions currently available on the market, which allow the formulation of water-sensitive active ingredients, while at the same time giving them good chemical stability, are generally compositions of salve type. These salve-type compositions consist mainly of petroleum jelly, and are called oleaginous salves. Now, petroleum jelly gives the product a very greasy and non-cosmetic feel, and leaves a greasy residue on the skin.

This greasy residue prevents the patient suffering from psoriasis from being able to get dressed again after treatment without the risk of leaving greasy marks on his or her clothing, which does not necessarily prompt the patient to follow his or her treatment. Non-compliant with the prescribed treatment is one of the main causes of failure; the article “Patients with psoriasis and their compliant with medication, Richards et al., J. Am. Acad. Dermatol., October 1999, p. 581-583” indicates that close to 40% of patients with a chronic disease such as psoriasis do not adhere to their treatment. Furthermore, the characteristics of the carrier used in the pharmaceutical compositions are directly linked to the patients' compliant with their treatment.

The compositions of oleaginous salve type currently on the market do not always lend themselves to the formulation of the active ingredient in a solubilized form.

EP-0,255,369 and U.S. Pat. No. 6,103,250 describe formulations most commonly based on silicone derivatives in which the water-sensitive active substances are conditioned in a dispersed form. However, the dispersed form is generally prejudicial to optimal release and/or penetration of these bioactive substances in the skin.

### SUMMARY OF THE INVENTION

The present invention features anhydrous pharmaceutical compositions suited for topical application and which make it possible to overcome the abovementioned drawbacks and disadvantages.

The present invention thus provides anhydrous pharmaceutical compositions suited for topical application, which exhibit sustained stability and the active ingredient of which is in a solubilized form.

Thus, this invention also features anhydrous pharmaceutical compositions useful in the treatment of psoriasis, comprising an organopolysiloxane elastomer and, as bioactive ingredient, at least one vitamin D-derived compound, said compound being in a solubilized form in said composition.

The vitamin D derivatives according to the invention are described in WO 03/050067. These are compounds which are structurally analogues of vitamin D, which show a selective activity on cell proliferation and differentiation, and which have a therapeutic activity with respect to dermatological conditions or skin disorders such as, for example, psoriasis.

The present invention therefore features anhydrous pharmaceutical compositions, which comprise an organopolysiloxane elastomer and, as bioactive agent, at least one vitamin D-derived compound in a solubilized form in said composition, said vitamin D derivative corresponding to general formula (I) below:

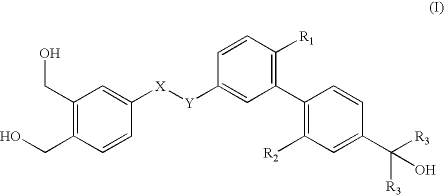

in which:

—X—Y— represents a link selected from among the following structures:

### —CH₂—CH₂

### —CH₂—O—

### —O—CH₂

### —CH₂—N(R₄)—

R₄ having the definition below,

R₁ is a methyl radical or an ethyl radical,

R₂ is an ethyl radical, a propyl radical or an isopropyl radical,

R₃ is an ethyl radical or a trifluoromethyl radical,

R₄ is a hydrogen atom, a methyl radical, an ethyl radical or a propyl radical.

The term “solubilized form” means a dispersion in the molecular state in a liquid, no crystallization of the active agent being visible to the naked eye or even under a cross-polarization optical microscope.

The compositions of the invention are more particularly useful for topical application.

The compositions of the invention are in particular useful in the treatment of psoriasis, whether it is cutaneous, mucosal or ungual, and of other skin disorders. The term “other skin disorders” means in particular dermatological conditions or afflictions with an inflammatory immunoallergic component, with or without a cell proliferation problem, such as psoriatic rheumatism or skin atopy, such as eczema.

Preferably, the compositions contain a single vitamin D-derived compound of formula (I).

The active ingredient of formula (I) is in the solubilized state, which confers on the compositions of the invention good properties of release/penetration into the skin of the active ingredient, allied with more advantageous kinetics. The expression “good release/penetration capacity” means a good distribution of the composition of the invention and therefore of the active ingredient that it contains, through the stratum corneum of the skin and also through the subcutaneous layers such as the epidermis and dermis.

For the purpose of the present invention, the expression “anhydrous composition” means a composition which is substantially free of water, i.e., which has a water content of less than or equal to 5% by weight relative to the total weight of the composition, in particular less than or equal to 3%, and especially equal to zero.

### DETAILED DESCRIPTION OF BEST MODE AND SPECIFIC/PREFERRED EMBODIMENTS OF THE INVENTION

According to an advantageous embodiment of the invention, the composition is in the form of a salve or of an ointment.

Preferably, the compositions of the invention are in the form of a salve. For the purposes of the present invention and according to US pharmacopoeia (“USP”), the term “salve” means a semi-solid preparation intended for external application to the skin or the mucous membranes. Salves or ointments (of softer consistency than salves) are administered topically for many applications, for example as barrier creams, antiseptics, emollients, etc. Salves are used for their emollient effect; they are simple to apply and readily penetrate into the skin.

Advantageously, the compositions according to the invention are found, after application, to be devoid of any tacky, greasy and shiny effect, and, on the other hand, provide a soft feel. This new type of salve improves absorption through the skin, leaves a powdery, nongreasy residue and is easy to apply, thereby improving the patient's compliant with his or her treatment. Moreover, an advantageous aspect of this composition is the absence of preservative.

In addition, the compositions are found to be particularly effective for preserving a satisfactory chemical stability of active ingredients sensitive to oxidation, to water and to aqueous media in general. The term “satisfactory chemical stability” means a composition which, over the course of a period of at least three months, respectively at ambient temperature and at 40° C.:

does not show any substantial modification of its macroscopic appearance,

comprises an active ingredient content of at least 90%, and more particularly of at least 95%, of the initial content by weight.

Among the compounds of formula (I) that are useful active ingredients in the present invention, exemplary are:
1. {5-[4′-(1-ethyl-1-hydroxypropyl)-6-methyl-2′-propylbiphenyl-3-yloxymethyl]-2-hydroxymethylphenyl}methanol;
2. {5-[6,2′-diethyl-4′-(1-ethyl-1-hydroxypropyl)biphenyl-3-yloxymethyl]-2-hydroxymethylphenyl}methanol;
3. {4-[6-ethyl-4′-(1-ethyl-1-hydroxypropyl)-2′-propylbiphenyl-3-yloxymethyl]-2-hydroxymethylphenyl}methanol;
4. {4-[6-ethyl-4′-(1-ethyl-1-hydroxypropyl)-2′-isopropylbiphenyl-3-yloxymethyl]-2-hydroxymethylphenyl}methanol;
5. (4-{2-[4′-(1-ethyl-1-hydroxypropyl)-6-methyl-2′-propylbiphenyl-3-yl]ethyl}-2-hydroxymethylphenyl)methanol;
6. {4-[4′-(1-ethyl-1-hydroxypropyl)-6-methyl-2′-propylbiphenyl-3-ylmethoxy]-2-hydroxymethylphenyl}methanol;
7. (4-{[4′-(1-ethyl-1-hydroxypropyl)-6-methyl-2′-propylbiphenyl-3-ylamino]methyl}-2-hydroxymethylphenyl)methanol;
8. [4-({[4′-(1-ethyl-1-hydroxypropyl)-6-methyl-2′-propylbiphenyl-3-yl]methylamino}methyl)-2-hydroxymethylphenyl]methanol;
9. [4-({ethyl-[4′-(1-ethyl-1-hydroxypropyl)-6-methyl-2′-propylbiphenyl-3-yl]amino}methyl)-2-hydroxymethylphenyl]methanol;
10. [4-({[4′-(1-ethyl-1-hydroxypropyl)-6-methyl-2′-propylbiphenyl-3-yl]propylamino}methyl)-2-hydroxymethylphenyl]methanol;
11. (2-hydroxymethyl-4-{2-[6-methyl-2′-propyl-4′-(2,2,2-trifluoro-1-hydroxy-1-trifluoromethylethyl)biphenyl-3-yl]ethyl}phenyl)methanol;
12. {2-hydroxymethyl-4-[6-methyl-2′-propyl-4′-(2,2,2-trifluoro-1-hydroxy-1-trifluoromethylethyl)biphenyl-3-yloxymethyl]phenyl}methanol;
13. {2-hydroxymethyl-4-[6-methyl-2′-propyl-4′-(2,2,2-trifluoro-1-hydroxy-1-trifluoromethylethyl)biphenyl-3-ylmethoxy]phenyl}methanol;
14. (2-hydroxymethyl-4-{[6-methyl-2′-propyl-4′-(2,2,2-trifluoro-1-hydroxy-1-trifluoromethylethyl)biphenyl-3-ylamino]methyl}phenyl)methanol;
15. [2-hydroxymethyl-4-({N-methyl-[6-methyl-2′-propyl-4′-(2,2,2-trifluoro-1-hydroxy-1-trifluoromethylethyl)biphenyl-3-yl]amino}methyl)phenyl]methanol;
16. [4-({N-ethyl-[6-methyl-2′-propyl-4′-(2,2,2-trifluoro-1-hydroxy-1-trifluoromethylethyl)biphenyl-3-yl]amino}methyl)-2-hydroxymethylphenyl]methanol;
17. [2-hydroxymethyl-4-({[6-methyl-2′-propyl-4′-(2,2,2-trifluoro-1-hydroxy-1-trifluoromethylethyl)biphenyl-3-yl]N-propylamino}methyl)phenyl]methanol;
18. (4-{2-[6-ethyl-4′-(1-ethyl-1-hydroxypropyl)-2′-propylbiphenyl-3-yl]ethyl}-2-hydroxymethylphenyl)methanol;
19. {4-[6-ethyl-4′-(1-ethyl-1-hydroxypropyl)-2′-propylbiphenyl-3-ylmethoxy]-2-hydroxymethylphenyl}methanol;
20. (4-{[6-ethyl-4′-(1-ethyl-1-hydroxypropyl)-2′-propylbiphenyl-3-ylamino]methyl}-2-hydroxymethylphenyl)methanol;
21. [4-({[6-ethyl-4′-(1-ethyl-1-hydroxypropyl)-2′-propylbiphenyl-3-yl]methylamino}methyl)-2-hydroxymethylphenyl]methanol;
22. [4-({ethyl-[6-ethyl-4′-(1-ethyl-1-hydroxypropyl)-2′-propylbiphenyl-3-yl]amino}methyl)-2-hydroxymethylphenyl]methanol;
23. [4-({[6-ethyl-4′-(1-ethyl-1-hydroxypropyl)-2′-propylbiphenyl-3-yl]propylamino}methyl)-2-hydroxymethylphenyl]methanol;
24. (4-{2-[6-ethyl-2′-propyl-4′-(2,2,2-trifluoro-1-hydroxy-1-trifluoromethylethyl)biphenyl-3-yl]ethyl}-2-hydroxymethylphenyl)methanol;
25. {4-[6-ethyl-2′-propyl-4′-(2,2,2-trifluoro-1-hydroxy-1-trifluoromethylethyl)biphenyl-3-yloxymethyl]-2-hydroxymethylphenyl}methanol;
26. {4-[6-ethyl-2′-propyl-4′-(2,2,2-trifluoro-1-hydroxy-1-trifluoromethylethyl)biphenyl-3-ylmethoxy]-2-hydroxymethylphenyl}methanol;
27. (4-{[6-ethyl-2′-propyl-4′-(2,2,2-trifluoro-1-hydroxy-1-trifluoromethylethyl)biphenyl-3-ylamino]methyl}-2-hydroxymethylphenyl)methanol;
28. [4-({[6-ethyl-2′-propyl-4′-(2,2,2-trifluoro-1-hydroxy-1-trifluoromethylethyl)biphenyl-3-yl]methylamino}methyl)-2-hydroxymethylphenyl]methanol;
29. [4-({N-ethyl[6-ethyl-2′-propyl-4′-(2,2,2-trifluoro-1-hydroxy-1-trifluoromethylethyl)biphenyl-3-yl]amino}methyl)-2-hydroxymethylphenyl]methanol;
30. [4-({[6-ethyl-2′-propyl-4′-(2,2,2-trifluoro-1-hydroxy-1-trifluoromethylethyl)biphenyl-3-yl]-N-propylamino}methyl)-2-hydroxymethylphenyl]methanol;
31. (4-{[4′-(1-ethyl-1-hydroxypropyl)-6,2′-dimethylbiphenyl-3-ylamino]methyl}-2-hydroxymethylphenyl)methanol.

Particularly preferably, the vitamin D derivative according to the present invention is the compound {4-[6-ethyl-4′-(1-ethyl-1-hydroxypropyl)-2′-propylbiphenyl-3-yloxymethyl]-2-hydroxymethylphenyl}methanol (compound 3 above), of formula (II) below:

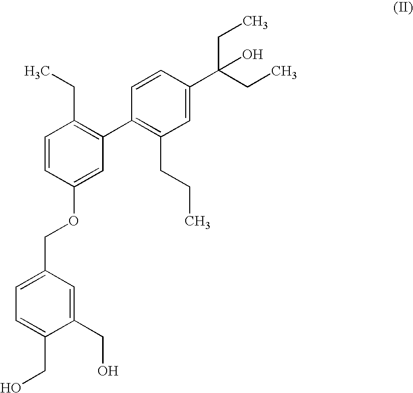

Advantageously, the amount of vitamin D derivative in a solubilized form in the compositions of the invention is from 0.00001% to 5% by weight relative to the total weight of the composition, preferably from 0.0001% to 3% by weight, and more particularly from 0.0003% to 1% by weight.

According to an advantageous embodiment of the invention, the active ingredient is solubilized in a solvent.

The solvent of the present invention is selected from among pharmaceutically acceptable compounds, i.e., compounds whose use is in particular compatible with application to the skin, the mucous membranes and/or the keratin fibers. It is generally pasty or fluid, and in particular liquid, at ambient temperature and ambient atmospheric pressure.

By way of solvent agents according to the invention, particularly exemplary are alcohols, in particular a pure alcohol, such as absolute ethanol, but also oleyl macrogol 6 glycerides known as Labrafil M1944CS marketed by Gattefossé, macrogol 15 hydroxystearate marketed under the trademark Solutol HS15 by BASF, PEG 40 hydrogenated castor oil marketed under the trademark Cremophor RH 40 by BASF, PEG 400 marketed under the trademark Lutrol E400 by BASF, dimethyl isosorbide marketed under the trademark Arlasolve DMI by Uniqema, PPG 15 stearyl ether marketed under the trademark Arlamol E by Uniqema, ethoxydiglycol marketed under the trademark Transcutol by Gattefossé, N-methyl-2-pyrrolidone marketed under the trademark Pharmasolve by ISP, and mixtures thereof.

Preferably, absolute ethanol or even a mixture of absolute ethanol with at least one of the above compounds, being called cosolvent(s), is preferably employed.

The solvent agent is generally present in the compositions of the invention in an amount, firstly, sufficient to provide the required solubility of the active ingredient to be formulated and, secondly, compatible with the need to preserve a sustained chemical stability of this same active ingredient. In other words, the solvent agent must be chemically inert with respect to the active ingredient.

Advantageously, the amount of solvent ranges from 1% to 25% by weight relative to the total weight of the composition, preferably from 1% to 20% by weight, preferably from 1% to 15% by weight.

Preferably, the absolute ethanol is used in an amount of from 1% to 6% by weight relative to the total weight of the composition, preferably from 4% to 6% by weight, while the amount of cosolvent(s) is from 1% to 15% by weight relative to the total weight of the composition, more particularly from 1% to 10% by weight.

The solvent agent also confers a beneficial effect on the degree of penetration of the active ingredients into the skin.

According to the invention, the composition comprises an organopolysiloxane elastomer, in a predominant amount by weight relative to the total weight of the composition.

The term “organopolysiloxane elastomer” means any chemically crosslinked siloxane polymer which has viscoelastic properties. According to the invention, such a polymer is non-reactive and non-polar.

Moreover, the elastomer according to the invention does not have an adhesive property. The term “adhesive” means a soft material which, when placed in the form of a thin film from two objects, makes it difficult to separate them; it is necessary to exert a certain amount of force in order to initiate the separation, and to provide, to the entirety, a certain amount of energy.

The term “viscoelastic properties” means the ability of the elastomer to deform up to a certain point, when subjected to a mechanical load, and to return to its original shape once said load has been removed.

As organopolysiloxane elastomers that can be formulated into the compositions of the invention, exemplary are those which are, in particular, described in U.S. Pat. Nos. 4,980,167 and 4,742,142. They may in particular be compounds resulting from addition reactions, i.e., hydrosilylation products or polymerization products derived from the addition of an organopolysiloxane having unsaturated groups, such as vinyl or allyl groups, in particular bonded to at least one terminal Si atom, with another silicone compound capable of participating in the addition reaction, such as an organohydrogenopolysiloxane.

Exemplary silicone elastomers are those prepared by means of a crosslinking reaction from polysiloxanes (A) containing ≡Si—H groups as defined below, with an alpha,omega-diene (B), in the presence of a catalyst and of a silicone oil (C).

The polysiloxane (A) containing the —Si—H unit can be represented by the compounds of formula R₃¹⁴SiO(R¹⁵₂SiO)ₐ(R¹⁶HSiO)_{b}SiR₃¹⁴, denoted herein as type A¹, and the compounds of formula HR₂¹⁴SiO(R¹⁵₂SiO)_{c}SiR₂¹⁴H or of formula HR₂¹⁴SiO(R¹⁵₂SiO)ₐ(R¹⁶HSiO)_{b}SiR₂¹⁴H, denoted herein as type A². In these formulae, R¹⁴, R¹⁵ and R¹⁶ are alkyl radicals containing from one to six carbon atoms, a is an integer ranging from 0 to 250, b is an integer ranging from 1 to 250 and c is an integer ranging from 0 to 250. The molar ratio of the compounds A²:A¹ is from 0 to 20, in particular from 0 to 5.

The alpha,omega-diene (B) is a compound of formula CH₂═CH(CH₂)_{d}CH═CH₂ in which d is an integer ranging from 1 to 20. Representative examples of suitable alpha,omega-dienes are 1,4-pentadiene, 1,5-hexadiene, 1,6-heptadiene, 1,7-octadiene, 1,8-nonadiene, 1,9-decadiene, 1,11-dodecadiene, 1,13-tetradecadiene and 1,19-elcosadiene.

The volatile or non-volatile silicone oil (C), in which the organopolysiloxane elastomer is formulated, is a low-molecular-weight polysiloxane and encompasses:

linear, cyclic or branched, low-molecular-weight volatile methyl siloxanes,

volatile or non-volatile, linear or cyclic, low-molecular-weight alkyl siloxanes and aryl siloxanes, and

linear or cyclic, low-molecular-weight functional siloxanes, and mixtures thereof.

The term “volatile” silicone oil means any silicone oil capable of evaporating on contact with the skin, the mucous membranes and the keratin fibers in less than one hour, at ambient temperature and atmospheric pressure.

Advantageously, the silicone oil (C) is a linear or cyclic, volatile polyorganosiloxane oil with a viscosity of less than or equal to 6 centistokes (6×10⁻⁶ m²/s), preferably containing from 2 to 10 silicon atoms, these silicones optionally comprising alkyl or alkoxy radicals containing from 1 to 22 carbon atoms.

More preferably, the silicone oil (C) is selected from among linear or cyclic, low-molecular-weight volatile methyl siloxanes.

Exemplary linear volatile methyl siloxanes are hexamethyldisiloxane, octamethyltrisiloxane, decamethyltetrasiloxane, dodecamethylpentasiloxane, tetradecylmethylhexasiloxane, hexadecamethylheptasiloxane, heptamethylhexyltrisiloxane and heptamethyloctyltrisiloxane.

Exemplary cyclic volatile methyl siloxanes are decamethylcyclopentasiloxane, hexamethylcyclotrisiloxane and dodecamethylcyclohexasiloxane.

Exemplary branched volatile methyl siloxanes are heptamethyl-3-[(trimethylsilyl)oxy]trisiloxane, hexamethyl-3,3-bis[(trimethylsilyl)oxy]trisiloxane and pentamethyl[(trimethylsilyl)oxy]cyclotrisiloxane.

Also suitable as oils (C) in the present invention are non-volatile, low-molecular-weight polysiloxanes corresponding to general formula:

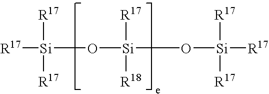

in which:

e is such that the polymers corresponding to this formula have a viscosity in the range of approximately 100 to 1000 centistokes (mm²/sec) and which is in particular selected in the range of from 80 to 375, R¹⁷ and R¹⁸ are alkyl radicals containing from 1 to 20 carbon atoms or an aryl radical such as a phenyl radical.

Among these polysiloxanes, particularly exemplary are polydimethylsiloxane, polydiethylsiloxane, polymethylethylsiloxane, polymethylphenylsiloxane and polydiphenylsiloxane.

Low-molecular-weight functionalized polysiloxanes can be represented by fluid siloxanes bearing acrylamide, acrylate, amide, amino, carbinol, carboxy, chloroalkyl, epoxy, glycol, ketal, mercapto, methyl ester, perfluoro and silanol functions.

Examples of elastomers thus obtained by crosslinking from (A) and (B) in the presence of (C) are in particular described in U.S. Pat. No. 5,654,362.

Preferably, the organopolysiloxane elastomer formulated in the compositions of the invention is in particular “ST Elastomer 10®” from Dow Corning, which is a copolymer of dimethicone/cyclomethicone formulated in a decamethylcyclopentasiloxane oil which is in the form of a thick translucent gel.

This type of elastomer is synthesized by means of a crosslinking reaction similar to that described above, i.e., prepared by means of a crosslinking reaction from polysiloxanes (A) containing ≡-SI—H groups as defined above, with an alpha,omega-diene (B), in the presence of a catalyst and of a linear or cyclic, low-molecular-weight polysiloxane (silicone oil (C)), to which vinyl siloxanes (or vinyl silanes) (A′) containing —CH═CH₂ vinyl groups are added.

Indeed, it has been demonstrated that the addition of these vinylsiloxanes (or vinyl silanes) blocks the remaining SiH functions which have not reacted (“quenching agent”). The compounds (A′) that can be used for the preparation of the preferred silicone agents according to the invention are such as those described in U.S. Pat. No. 5,929,164. By way of examples of such vinyl siloxane or vinyl silane compounds (A′), representative are vinyl-t-butyldimethylsilane, vinyidiethylmethylsilane, vinylethyldimethylsilane, vinyltriethylsilane, vinyltrimethylsilane, divinyldimethylsilane, divinyltetramethyldisilane, vinylpentamethyldisiloxane, 1,3-divinyltetramethyldisiloxane, a vinyltrisiloxane of structure (CH₃)₃SiOSi(CH═CH₂) (CH₃)OSi(CH₃)₃, 1,5-divinylhexamethyltrisiloxane, and a divinylsiloxane oligomer having a structure (CH₂═CH)Me₂SiO(Me₂SiO)₈SiMe₂(CH═CH₂).

The alpha,omega-diene (B) preferred according to the invention is 1,5-hexadiene.

According to a specific embodiment, the silicone agents according to the invention are preferentially polysiloxane elastomers which do not contain a hydrophilic group. According to the invention, the term “hydrophilic group” means, for example, a group of polyoxyalkylene type or a group of glycol type.

The silicone elastomer defined above can serve in particular the function of thickener in the compositions according to the invention. It can also contribute to their stabilization.

Advantageously, the amount of organopolysiloxane elastomer, which is predominant in the compositions according to the invention, can vary substantially, in particular according to the desired viscosity of the composition.

The term “amount of organopolysiloxane elastomer” means the amount of elastomer and of silicone oil in which it is formulated.

Advantageously, the amount of organopolysiloxane elastomer in a composition of the invention is from 70% to 95% by weight relative to the total weight of the composition, preferably from 80% to 95% by weight, more preferably from 83% to 92% by weight.

The compositions can also comprise, in addition to the silicone oil (C) in which the organopolysiloxane elastomer is formulated, at least one additional silicone oil. Among these additional silicone oils, exemplary is cyclomethicone and/or dimethicone having a viscosity from 20 to 350 centistokes. These oils are used in an amount of from 1% to 30% by weight, preferably from 1% to 15% by weight.

According to another advantageous embodiment, the compositions also comprise a feel additive. The term “feel additive” means a compound introduced into a composition in order to improve the feel thereof on the skin.

Such an additive is in particular selected from the group consisting of:

the isopropyl palmitate known under the trademark Crodamol IPP, the isopropyl myristate known under the trademark Crodamol IPM, C₁₂-C₁₅ alkyl benzoate, hexyl laurate, diisopropyl adipate, isononyl isononanoate, 2-ethylhexyl palmitate, isostearyl isostearate, octanoates, decanoates or ricinoleates of alcohols or polyalcohols, such as propylene glycol dioctanoate, and mixtures thereof;

hydroxylated esters, such as isostearyl lactate, diisostearyl malate, pentaerythritol esters, and mixtures thereof;

non-volatile oils of formula R²¹CO—OR²² in which R²¹ and R²² each independently represent a C₁ to C₂₅, in particular C₄ to C₂₀, linear or branched alkyl radical or alkenyl, alkoxycarbonylalkyl or alkylcarbonyloxyalkyl radicals. Examples of such esters include isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coco-caprate/dicaprylate, decyl isostearate, isodecyl oleate, isodecyl neopentanoate, isohexyl neopentanoate, octyl palmitate, dioctyl malate, tridecyl octanoate, myristyl myristate and octododecanol.

The addition of a feel additive makes it possible to prevent fluffing. Preferentially, isopropyl palmitate, isopropyl myristate, C₁₂-C₁₅ alkyl benzoate, or mixtures thereof, will be used.

Preferably, the compositions according to the invention comprise:

from 70% to 95% of organopolysiloxane elastomer;

from 0% to 26% of a silicone oil;

from 0% to 15% of cosolvent;

from 0% to 10% of absolute ethanol;

from 0% to 5% of a feel additive;

from 0.0001% to 0.5% of active ingredient of formula (I).

Preferentially, the compositions comprise:

from 80% to 95% of organopolysiloxane elastomer;

from 0% to 15% of a silicone oil;

from 0% to 12% of cosolvent;

from 0% to 6% of absolute ethanol;

from 0% to 3% of a feel additive;

from 0.0001% to 0.3% of active ingredient of formula (I).

Even more preferentially, the compositions comprise:

from 83% to 92% of organopolysiloxane elastomer;

from 0% to 5% of a silicone oil;

from 0% to 9% of cosolvent;

from 4% to 6% of absolute ethanol;

from 1% to 2% of a feel additive;

from 0.001% to 0.3% of active ingredient of formula (I).

It should be noted that the compositions according to the invention do not comprise any wax or additional thickener since this causes physical instability of the formulae.

The compositions according to the invention can, however, comprise various other ingredients. The selection of these additional ingredients, just as that of their respective amounts, is made so as not to adversely affect the properties expected for the composition. In other words, these compounds should not affect the chemical stability of the active ingredients, nor their solubility.

According to an advantageous embodiment of the invention, the composition also comprises an anti-oxidizing agent selected from the group consisting of butylhydroxytoluene (BHT), butylhydroxyanisole (BHA), DL-alpha-tocopherol and propyl gallate.

The composition can also comprise a lipophilic anti-irritant selected from the group consisting of DL-alpha-tocopherol acetate, tea tree oil, green tea extract and calendula extract.

Of course, one skilled in the art will take care to choose the optional compound(s) to be added to these compositions in such a way that the advantageous properties intrinsically associated with the present invention are not or not substantially impaired by the envisaged addition.

The present invention also features the formulation of an organopolysiloxane elastomer into an anhydrous pharmaceutical composition useful in the treatment of psoriasis, whether regime or regimen, said composition comprising, as active ingredient, at least one vitamin D derivative of general formula (I), said active ingredient being in a solubilized form.

In the application indicated above, the pharmaceutical composition is as defined above.

Finally, the present invention features a process for preparing a composition as described above, which comprises mixing at least two distinct phases: a phase comprising at least the organopolysiloxane elastomer, and a phase comprising at least the active ingredient and the solvent, optionally the cosolvent(s), of said active ingredient.

The process comprises the following steps:

a) preparing phase A, which comprises the organopolysiloxane elastomer, optionally mixed with an additional silicone oil and/or a feel additive, to homogeneity;

b) preparing phase B, by mixing at least one vitamin D derivative of general formula (I) with the solvent, to homogeneity;

c) incorporating phase B into phase A, and then homogenizing until a homogeneous gel is obtained.

The solvent of step b) is preferably ethanol.

The process can optionally comprise, at the beginning of step b), a step of mixing the various cosolvents in ethanol, before introducing the vitamin D-derived active agent.

According to an advantageous embodiment of the invention, the process is carried out under cold conditions, i.e., at ambient temperature from 20° C. to 25° C. This is also the reason why the compositions according to the invention contain neither wax nor additional thickener, since, if such compounds are introduced, the process can no longer be carried out under cold conditions.

In order to further illustrate the present invention and the advantages thereof, the following specific examples are given, it being understood that same are intended only as illustrative and in nowise limitative. In said examples to follow, all parts and percentages are given by weight, unless otherwise indicated.

### Example 1

### Active Ingredient Solubility Tests

The solubility of {4-[6-ethyl-4′-(1-ethyl-1-hydroxypropyl)-2′-propylbiphenyl-3-yloxymethyl]-2-hydroxymethylphenyl}methanol was tested in various solvents.

| Excipients | Max sol (% w/w) |
|---|---|
| Propylene Glycol | 2.3351 |
| Ethanol 95 | >20 |
| PEG 400 | 6.894 |
| Transcutol | >20 |
| Sweet almond oil | 0.0932 |
| Cremophor RH40 | 3.989 |
| Arlamol E | 1.033 |
| Labrafil M1944CS | 0.936 |
| Eutanol G | 0.322 |
| Miglyol 812 | 0.3167 |
| IPP | 0.1654 |
| Mirasil CM5 | NA |
| Primol 352 | 0.0009 |

### Example 2

### Formulations in Accordance with the Invention

a) Composition 1:

| Phases | INCI name | Formulary % |
|---|---|---|
| A | Cyclomethicone & dimethicone crosspolymer | 74.8 |
| A | Cyclomethicone 5 | 18.0 |
| A | Isopropyl palmitate | 1.00 |
| A | DL-alpha-tocopherol acetate | 1.00 |
| A | DL-alpha-tocopherol | 0.10 |
| B | Ethanol 100 | 5.00 |
| B | {4-[6-Ethyl-4′-(1-ethyl-1-hydroxypropyl)-2′- | 0.10 |
| | propylbiphenyl-3-yloxymethyl]-2- | |
| | hydroxymethylphenyl}methanol | |

b) Composition 2:

| Phases | INCI name | Formulary % |
|---|---|---|
| A | Cyclomethicone & dimethicone crosspolymer | 84.9 |
| A | Isopropyl palmitate | 1.00 |
| A | DL-alpha-tocopherol | 0.10 |
| B | PEG 8 | 5.00 |
| B | Oleyl macrogol 6 glycerides | 2.9 |
| B | PEG 40 castor oil hydrogenated | 1.00 |
| B | Ethanol 100 | 0.10 |
| B | {4-[6-Ethyl-4′-(1-ethyl-1-hydroxypropyl)-2′- | 0.10 |
| | propylbiphenyl-3-yloxymethyl]-2- | |
| | hydroxymethylphenyl}methanol | |

c) Composition 3:

| Phases | INCI name | Formulary % |
|---|---|---|
| A | Cyclomethicone & dimethicone crosspolymer | 91.8 |
| A | Isopropyl palmitate | 1.00 |
| A | DL-alpha-tocopherol | 0.10 |
| B | Macrogol 15 hydroxystearate | 2.00 |
| B | Ethanol 100 | 5.00 |
| B | {4-[6-Ethyl-4′-(1-ethyl-1-hydroxypropyl)-2′- | 0.10 |
| | propylbiphenyl-3-yloxymethyl]-2- | |
| | hydroxymethylphenyl}methanol | |

Procedure for Compositions 1, 2 and 3:

Production at ambient temperature under inactinic light.

Phase Preparation:

Phase A:

The starting materials are homogenized in the formulary beaker, with stirring using a rayneri mixer (deflocculating paddle).

Phase B (Active Phase):

The various solvents are homogenized in ethanol, with magnetic stirring, and then the active agent {4-[6-ethyl-4′-(1-ethyl-1-hydroxypropyl)-2′-propylbiphenyl-3-yloxymethyl]-2-hydroxymethylphenyl}methanol) is introduced and the stirring is maintained until the active agent is completely solubilized.

The active phase is incorporated into the gel formed, with stirring in a rayneri mixer, and the mixture is homogenized until a homogeneous gel is obtained.

d) Composition 4:

| Phases | INCI name | Formulary % |
|---|---|---|
| A | Cyclomethicone & dimethicone crosspolymer | 84.8 |
| A | Isopropyl palmitate | 1.00 |
| B | PEG 8 | 5.00 |
| B | Oleyl macrogol 6 glycerides | 1.00 |
| B | Hydrogenated PEG 40 castor oil | 3.00 |
| B | Butylhydroxytoluene | 0.10 |
| B | Ethanol 100 | 5.00 |
| B | {4-[6-Ethyl-4′-(1-ethyl-1-hydroxypropyl)-2′- | 0.10 |
| | propylbiphenyl-3-yloxymethyl]-2- | |
| | hydroxymethylphenyl}methanol | |

e) Composition 5:

| Phases | INCI name | Formulary % |
|---|---|---|
| A | Cyclomethicone & dimethicone crosspolymer | 89.8 |
| A | Isopropyl palmitate | 1.00 |
| B | PPG 15 stearyl ether | 2.00 |
| B | Dimethyl isosorbide | 2.00 |
| B | DL-alpha-tocopherol | 0.10 |
| B | Ethanol 100 | 5.00 |
| B | {4-[6-Ethyl-4′-(1-ethyl-1-hydroxypropyl)-2′- | 0.10 |
| | propylbiphenyl-3-yloxymethyl]-2- | |
| | hydroxymethylphenyl}methanol | |

f) Composition 6:

| Phases | INCI name | Formulary % |
|---|---|---|
| A | Cyclomethicone & dimethicone crosspolymer | 83.8 |
| A | Isopropyl palmitate | 1.00 |
| B | PEG 8 | 5.00 |
| B | Hydrogenated PEG 40 castor oil | 3.00 |
| B | PPG 15 stearyl ether | 2.00 |
| B | Butylhydroxytoluene | 0.10 |
| B | Ethanol 100 | 5.00 |
| B | {4-[6-Ethyl-4′-(1-ethyl-1-hydroxypropyl)-2′- | 0.10 |
| | propylbiphenyl-3-yloxymethyl]-2- | |
| | hydroxymethylphenyl}methanol | |

Procedure for compositions 4, 5 and 6:

Production at ambient temperature under inactinic light.

Phase Preparation:

Phase A:

The cyclomethicone dimethicone crosspolymer, the isopropyl palmitate and the DL-alpha-tocopherol (if present in the composition) are weighed out into the formulary beaker.

The mixture is homogenized with stirring in a rayneri mixer (deflocculating paddle).

Phase B (Active Phase):

The butylhydroxytoluene (if present in the composition) is solubilized in the ethanol and the various solvents, with magnetic stirring.

The active agent ({4-[6-ethyl-4′-(1-ethyl-1-hydroxypropyl)-2′-propylbiphenyl-3-yloxymethyl]-2-hydroxymethylphenyl}methanol) is introduced and the stirring is maintained until the active agent is completely solubilized.

The active phase is incorporated into the gel formed, with stirring in a rayneri mixer, and the mixture is homogenized until a homogeneous gel is obtained.

### Example 3

### Study of the Physical and Chemical Stabilities of the Formulations

a) Physical Stability:

The physical stability of the formulations is evaluated by macroscopic and microscopic observation of the formulation at ambient temperature, 40° C. and 40° C. at T1 month, T2 months and T3 months.

At AT the macroscopic observation makes it possible to guarantee the physical integrity of the products. The characterization of the finished product is completed by measurement of the flowing threshold.

A Haake VT550 rheometer is used with an SVDIN measuring spindle.

The rheograms are produced at 25° C. and at a shear rate of 4 s⁻¹ (γ), and by measuring the shear stress. The term “flow threshold” (τ0 expressed in Pascals) means the force necessary (minimum shear stress) to overcome the Van der Waals cohesion forces and to bring about flow. The flow threshold is comparable to the value found at the shear rate of 4 s⁻¹.

These measurements are carried out at T24 h, and at T1, T2 and T3 months.

b) Chemical Stability:

The active agent ({4-[6-ethyl-4′-(1-ethyl-1-hydroxypropyl)-2′-propylbiphenyl-3-yloxymethyl]-2-hydroxymethylphenyl}methanol) is assayed by HPLC at AT and 40° C. at TO, T1, T2 and T3 months.

Result obtained: R as %

Composition 1:

SPECIFICATIONS AT T0

| | | | | | |
|---|---|---|---|---|---|
| Analytical | Centrif- | 3000 rpm | NTR* | Macroscopic | Transparent |
| assay T0: | ugation | | | appearance | and fluid |
| 101% | | | | | gel |
| | | 10 000 rpm | exudate | Viscosity: | 56 |
| | | | | T(4 s⁻¹) in | |
| | | | | Pa · s⁻¹ | |

| | | T1 month | T2 months |
|---|---|---|---|
| AT | Viscosity: T_{(4 s}⁻¹₎ in | 53 | 55 |
| | Pa · s⁻¹ | | |
| | Macroscopic | Compliant | Compliant |
| | appearance | | |
| | Microscopic | Compliant | Compliant |
| | appearance | | |
| | Analytical assay | 99.3% | 100% |
| 4° C. | Macroscopic | Compliant | Compliant |
| | appearance | | |
| | Microscopic | Compliant | Compliant |
| | appearance | | |
| 40° C. | Macroscopic | Compliant | Compliant |
| | appearance | | |
| | Microscopic | Compliant | Compliant |
| | appearance | | |
| | Viscosity: T_{(4 s}⁻¹₎ in | / | 48 |
| | Pa · s⁻¹ | | |
| | Analytical assay | 99.6% | 99.8% |

*NTR: Nothing to report

Composition 2:

SPECIFICATIONS AT T0

| | | | | | |
|---|---|---|---|---|---|
| Analytical | Centrifugation | 3000 rpm | NTR | Macroscopic | Opaque |
| assay T0: | | | | appearance | gel |
| 103% | | | | | |
| | | 10 000 rpm | Light | Viscosity: | 96 |
| | | | pellet | T_{(4 s}⁻¹₎ in | |
| | | | | Pa · s⁻¹ | |

| | | T1 month | T2 months | T3 months |
|---|---|---|---|---|
| AT | Viscosity: T_{(4 s}⁻¹₎ in | 108 | 91 | 103 |
| | Pa · s⁻¹ | | | |
| | Macroscopic | Compliant | Compliant | Compliant |
| | appearance | | | |
| | Microscopic | Compliant | Compliant | Compliant |
| | appearance | | | |
| | Analytical assay | 101.2% | 101.5% | T25 weeks: |
| | | | | 98.8% |
| 4° C. | Macroscopic | Compliant | Compliant | Compliant |
| | appearance | | | |
| | Microscopic | Compliant | Compliant | Compliant |
| | appearance | | | |
| 40° C. | Macroscopic | Compliant | Compliant | Compliant |
| | appearance | | | |
| | Microscopic | Compliant | Slight | Yellow |
| | appearance | | yellowing | |
| | Viscosity: T_{(4 s}⁻¹₎ in | NA | NA | 111 |
| | Pa · s⁻¹ | | | |
| | Analytical assay | 101.1% | 104.7% | T25 weeks: |
| | | | | 98.1% |

Composition 3:

SPECIFICATIONS AT T0

| | | | | | |
|---|---|---|---|---|---|
| Analytical | Centrifugation | 3000 rpm | NTR | Macroscopic | Thick |
| assay T0: | | | | appearance | opaque |
| 102.4% | | | | | gel |
| | | 10 000 rpm | NTR | Viscosity: | 236 |
| | | | | T_{(4 s}⁻¹₎ in | |
| | | | | Pa · s⁻¹ | |

| | | T1 month | T2 months | T3 months |
|---|---|---|---|---|
| AT | Viscosity: T_{(4 s}⁻¹₎ in | 259 | 277 | 247 |
| | Pa · s⁻¹ | | | |
| | Macroscopic | Compliant | Compliant | Compliant |
| | appearance | | | |
| | Microscopic | Compliant | Compliant | Compliant |
| | appearance | | | |
| | Analytical assay | 101.5% | 101.5% | 101.9% |
| 4° C. | Macroscopic | Compliant | Compliant | Compliant |
| | appearance | | | |
| | Microscopic | Compliant | Compliant | Compliant |
| | appearance | | | |
| 40° C. | Macroscopic | Compliant | Compliant | Compliant |
| | appearance | | | |
| | Microscopic | Compliant | Compliant | Compliant |
| | appearance | | | |
| | Viscosity: T_{(4 s}⁻¹₎ in | NA | NA | 431 |
| | Pa · s⁻¹ | | | |
| | Analytical assay | 101.9% | 100.5% | 101% |

Composition 4:

SPECIFICATIONS AT T0

| | | | | | |
|---|---|---|---|---|---|
| Analytical | Centrifugation | 3000 rpm | NTR | Macroscopic | Opaque |
| assay T0: | | | | appearance | gel |
| 100.6% | | | | | |
| | | 10 000 rpm | NTR | Viscosity: | 118 |
| | | | | T_{(4 s}⁻¹₎ in | |
| | | | | Pa · s⁻¹ | |

| | | T1 month | T2 months | T3 months |
|---|---|---|---|---|
| AT | Viscosity: T_{(4 s}⁻¹₎ in | 88 | 116 | 104 |
| | Pa · s⁻¹ | | | |
| | Macroscopic | Compliant | Compliant | Compliant |
| | appearance | | | |
| | Microscopic | Compliant | Compliant | Compliant |
| | appearance | | | |
| | Analytical assay | 102.8% | 102.3% | 100.5% |
| 4° C. | Macroscopic | Compliant | Compliant | Compliant |
| | appearance | | | |
| | Microscopic | Compliant | Compliant | Compliant |
| | appearance | | | |
| 40° C. | Macroscopic | Compliant | Compliant | Compliant |
| | appearance | | | |
| | Microscopic | Compliant | Compliant | Compliant |
| | appearance | | | |
| | Viscosity: T_{(4 s}⁻¹₎ in | NA | NA | 122 |
| | Pa · s⁻¹ | | | |
| | Analytical assay | 103.2% | 101.5% | 101.3% |

Composition 5:

SPECIFICATIONS A T0

| | | | | | |
|---|---|---|---|---|---|
| Analytical | Centrifugation | 3000 rpm | NA | Macroscopic | Opaque |
| assay T0: | | | | appearance | gel |
| 100.6% | | | | | |
| | | 10 000 rpm | NA | Viscosity: | 185 |
| | | | | T_{(4 s}⁻¹₎ in | |
| | | | | Pa · s⁻¹ | |

Composition 6:

SPECIFICATIONS AT T0

| | | | | | |
|---|---|---|---|---|---|
| Analytical | Centrifugation | 3000 rpm | NA | Macroscopic | Opaque |
| assay T0: | | | | appearance | gel |
| 101.9% | | | | | |
| | | 10 000 rpm | NA | Viscosity: | 102 |
| | | | | T_{(4 s}⁻¹₎ in | |
| | | | | Pa · s⁻¹ | |

| | | T1 month | T2 months | T3 months |
|---|---|---|---|---|
| AT | Viscosity: T_{(4 s}⁻¹₎ in | 107 | 106 | 108 |
| | Pa · s⁻¹ | | | |
| | Macroscopic | Compliant | Compliant | Compliant |
| | appearance | | | |
| | Microscopic | Compliant | Compliant | Compliant |
| | appearance | | | |
| | Analytical assay | 102.8% | No assay | T19 weeks: |
| | | | | 104.7% |
| 4° C. | Macroscopic | Compliant | Compliant | Compliant |
| | appearance | | | |
| | Microscopic | Compliant | Compliant | Compliant |
| | appearance | | | |
| 40° C. | Macroscopic | Compliant | Compliant | Compliant |
| | appearance | | | |
| | Microscopic | Compliant | Compliant | Compliant |
| | appearance | | | |
| | Viscosity: T_{(4 s}⁻¹₎ in | NA | NA | 115 |
| | Pa · s⁻¹ | | | |
| | Analytical assay | 101.8% | No assay | T19 weeks: |
| | | | | 103% |

### Example 4

### Optimization of the Active-Phase Solubilization Time

a) Three active phases are prepared, two samples are taken from each of the phases after a defined stirring time: 15 min and 30 min.

| | Active | | |
|---|---|---|---|
| | phase | Active phase | Active phase |
| | composition 3 | composition 4 | composition 5 |
| Macrogol 15 hydroxystearate | 2% | | |
| (Solutol HS15) | | | |
| Oleyl macrogol 6 glycerides | | 1% | |
| Hydrogenated PEG 40 castor | | 3% | |
| oil | | | |
| PEG 8 | | 5% | |
| Dimethyl isosorbide (Arlasolve | | | 2% |
| DMI) | | | |
| PPG 15 stearyl ether (Arlamol | | | 2% |
| E) | | | |
| Butylhydroxytoluene | | 0.1% | |
| {4-[6-ethyl-4′-(1-ethyl-1- | 0.1% | 0.1% | 0.1% |
| hydroxypropyl)-2′- | | | |
| propylbiphenyl-3-yloxymethyl]- | | | |
| 2- | | | |
| hydroxymethylphenyl}methanol | | | |
| Ethanol | 5% | 5% | 5% |
| Procedure | Solubilization | Homogenization | Homogenization |
| | of Solutol | of all the | of the Arlasolve |
| | HS15 in | solvents in | DMI and of the |
| | ethanol then | ethanol and | Arlamol E in |
| | solubilization | then | ethanol and the |
| | of the active | solubilization of | solubilization of |
| | agent | the active agent | the active agent |
| Type of homogenization | Magnetic | Magnetic | Magnetic |
| Solvent homogenization time | 30 mn | 20 mn | 5 mn |
| before addition of compound A | | | |

| | |
|---|---|
| Amount produced | For 1.5 kg of finished product |
| Macroscopic and microscopic | 15′ of stirring: 1st sample taken |
| appearance of the active | Clear phase, absence of crystals |
| phase at AT | NTR 20d |
| | 30′ of stirring: 2nd sample taken |
| | NTR 20d |
| Macroscopic and microscopic | 15′ of stirring: 1st sample taken |
| appearance of the active | NTR 20d |
| phase after storage at 4° C. | 30′ of stirring: 2nd sample taken |
| | NTR 20d |

Conclusion:

This demonstrates that the {4-[6-ethyl-4′-(1-ethyl-1-hydroxypropyl)-2′-propylbiphenyl-3-yloxymethyl]-2-hydroxymethylphenyl}methanol is well solubilized after 15 minutes of stirring.

b) Composition 3 is thus produced with optimization of the solubilization time for {4-[6-ethyl-4′-(1-ethyl-1-hydroxypropyl)-2′-propylbiphenyl-3-yloxymethyl]-2-hydroxymethylphenyl}methanol in ethanol 100 and macrogol 15 hydroxystearate (15 min), and its physical stability is evaluated:

SPECIFICATIONS AT T0

| | | | | | |
|---|---|---|---|---|---|
| Analytical | Centrifugation | 3000 rpm | NA | Macroscopic | Thick |
| assay | | | | appearance | opaque |
| No assay | | | | | gel |
| | | 10 000 rpm | NA | Viscosity: | 261 |
| | | | | T_{(4 s}⁻¹₎ in | |
| | | | | Pa · s⁻¹ | |

| | | T1 month | T2 months | T3 months |
|---|---|---|---|---|
| AT | Viscosity: T_{(4 s}⁻¹₎ in | NA | NA | 248 |
| | Pa · s⁻¹ | | | |
| | Macroscopic | Compliant | Compliant | Compliant |
| | appearance | | | |
| | Microscopic | Compliant | Compliant | Compliant |
| | appearance | | | |
| 4° C. | Macroscopic | Compliant | Compliant | Compliant |
| | appearance | | | |
| | Microscopic | Compliant | Compliant | Compliant |
| | appearance | | | |
| 40° C. | Macroscopic | Compliant | Compliant | Compliant |
| | appearance | | | |
| | Microscopic | Compliant | Compliant | Compliant |
| | appearance | | | |
| | Viscosity: T_{(4 s}⁻¹₎ in | NA | NA | 290 |
| | Pa · s⁻¹ | | | |

Conclusion:

Good solubilization of the active agent, no recrystallization problem.

### Example 5

### Optimization of the Process

Placebo formulas are produced in a reactor (IKA LR 2.5T) (amount produced 1.5 kg), and their physical stability is evaluated.

a) Placebo composition 3:

| Phases | INCI name | Formulary % |
|---|---|---|
| A | Cyclomethicone & dimethicone crosspolymer | 91.8 |
| A | Isopropyl palmitate | 1.00 |
| A | DL-alpha-tocopherol | 0.10 |
| B | Macrogol 15 hydroxystearate | 2.00 |
| B | Ethanol 100 | 5.00 |

Procedure:

The following are successively introduced into the reactor tank:

cyclomethicone & dimethicone crosspolymer (stirring speed 7 rpm)

isopropyl palmitate+DL-alpha-tocopherol, previously homogenized with magnetic stirring for 10 min.

Stirring speed 26 rpm, vacuum: −0.7 bar.

The solvent phase is then slowly introduced: Absolute ethanol+macrogol 15 hydroxystearate (homogenized beforehand with magnetic stirring for 40 min).

Stirring speed 53 rpm then increase to 89 rpm at the end of the addition of the solvent phase for good homogenization.

Deaeration of the product before stirring, speed 7 rpm, vacuum: −0.8 bar for 50 min.

Physical Stability:

| | | T0 | T3 months |
|---|---|---|---|
| AT | Viscosity: T₍₄ₛ⁻¹₎ in Pa · s⁻¹ | 221 | 207 |
| | Macroscopic appearance | Thick opaque | Compliant |
| | | gel | |
| 40° C. | Macroscopic appearance | / | Compliant |
| | Viscosity: T₍₄ₛ⁻¹₎ in Pa · s⁻¹ | / | 221 |

b) Placebo composition 4:

| Phases | INCI name | Formulary % |
|---|---|---|
| A | Cyclomethicone & dimethicone crosspolymer | 84.9 |
| A | Isopropyl palmitate | 1.00 |
| B | PEG 8 | 5.00 |
| B | Hydrogenated PEG 40 castor oil | 3.00 |
| B | Oleyl macrogol 6 glycerides | 1.00 |
| B | Butylhydroxytoluene | 0.10 |
| B | Ethanol 100 | 5.00 |

Procedure:

The following are successively introduced into the reactor tank:

cyclomethicone & dimethicone crosspolymer (stirring speed 7 rpm) isopropyl palmitate.

Stirring speed 26 rpm, vacuum: −0.7 bar.

The solvent phase is then slowly introduced: Absolute ethanol+oleyl macrogol 6 glycerides+hydrogenated PEG 40 castor oil+PEG 8+butylhydroxytoluene (homogenized beforehand with magnetic stirring for 20 min).

Stirring speed 50 rpm then increase to 90 rpm at the end of the addition of the solvent phase for good homogenization.

The product is deaerated with slow stirring, speed 10 rpm, vacuum: −0.8 bar for 30 min.

Physical Stability:

| | | T0 | T3 months |
|---|---|---|---|
| AT | Viscosity: T₍₄ₛ⁻¹₎ in Pa · s⁻¹ | 102 | 83 |
| | Macroscopic appearance | Thick opaque | Compliant |
| | | gel | |
| 40° C. | Macroscopic appearance | / | Compliant |
| | Viscosity: T₍₄ₛ⁻¹₎ in Pa · s⁻¹ | / | 95 |

Conclusion:

better homogenization of the products by virtue of the stirring system (anchor shaft);

deaerated products obtained by virtue of the stirring under vacuum.

### Example 6

### Variation of the Concentration of Active Agent

a) Physical stability of composition 3 with variation of the concentration of {4-[6-ethyl-4′-(1-ethyl-1-hydroxypropyl)-2′-propylbiphenyl-3-yloxymethyl]-2-hydroxymethylphenyl}methanol from 0.15% to 0.3% by weight relative to the weight of the total composition (m/m):

| | | Concentration of {4-[6- | | | |
|---|---|---|---|---|---|
| | | ethyl-4′-(1-ethyl-1- | | | |
| | | hydroxypropyl)-2′- | | | |
| | | propylbiphenyl-3-yloxy- | | | |
| | | methyl]-2-hydroxy- | | | |
| | | methylphenyl}methanol | | | |
| | | 0.15% | 0.2% | 0.25% | 0.3% |
| AT | Macroscopic | Thick | Thick | Thick | Thick |
| T0 | appearance | opaque | opaque | opaque | opaque |
| | microscopic | gel | gel | gel | gel 207 |
| | appearance | 241 | 237 | 213 | |
| | Viscosity: | | | | |
| | T_{(4 s}⁻¹₎ in | | | | |
| | Pa · s⁻¹ | | | | |
| AT | Macroscopic | Compliant | Compliant | Compliant | Compliant |
| T3 m | appearance | 252 | 256 | 238 | 225 |
| | microscopic | | | | |
| | appearance | | | | |
| | Viscosity: | | | | |
| | T_{(4 s}⁻¹₎ in | | | | |
| | Pa · s⁻¹ | | | | |
| 4° C. | Macroscopic | Compliant | Compliant | Compliant | Compliant |
| T3 m | appearance | | | | |
| | microscopic | | | | |
| | appearance | | | | |
| 40° C. | Macroscopic | Compliant | Compliant | Compliant | Compliant |
| T3 m | appearance | 231 | 256 | 390 | 228 |
| | microscopic | | | | |
| | appearance | | | | |
| | Viscosity: | | | | |
| | T_{(4 s}⁻¹₎ in | | | | |
| | Pa · s⁻¹ | | | | |

b) Physical stability of composition 4 with variation of the concentration of {4-[6-ethyl-4′-(1-ethyl-1-hydroxypropyl)-2′-propylbiphenyl-3-yloxymethyl]-2-hydroxymethylphenyl}methanol from 0.15% to 0.3% (m/m):

| | | Concentration of {4-[6- | | | |
|---|---|---|---|---|---|
| | | ethyl-4′-(1-ethyl-1- | | | |
| | | hydroxypropyl)-2′- | | | |
| | | propylbiphenyl-3-yloxy- | | | |
| | | methyl]-2-hydroxy- | | | |
| | | methylphenyl}methanol | | | |
| | | 0.15% | 0.2% | 0.25% | 0.3% |
| AT | Macroscopic | Thick | Thick | Thick | Thick |
| T0 | appearance | opaque | opaque | opaque | opaque |
| | microscopic | gel | gel | gel | gel 86 |
| | appearance | 98 | 107 | 101 | |
| | Viscosity: | | | | |
| | T_{(4 s}⁻¹₎ | | | | |
| AT | Macroscopic | Compliant | Compliant | Compliant | Compliant |
| T3 m | appearance | 129 | 112 | 110 | 121 |
| | microscopic | | | | |
| | appearance | | | | |
| | Viscosity: | | | | |
| | T_{(4 s}⁻¹₎ | | | | |
| 4° C. | Macroscopic | Compliant | Compliant | Compliant | Compliant |
| T3 m | appearance | | | | |
| | microscopic | | | | |
| | appearance | | | | |
| 40° C. | Macroscopic | Compliant | Compliant | Compliant | Compliant |
| T3 m | appearance | 114 | 131 | 121 | 121 |
| | microscopic | | | | |
| | appearance | | | | |
| | Viscosity: | | | | |
| | T_{(4 s}⁻¹₎ | | | | |

The compositions are therefore physically stable for active agent concentrations of from 0.15% to 0.3% (m/m).

### Example 5

### Local Tolerance Study

A tolerance study was carried out on placebos of reference formulations and of composition 2.

Treatment: Daily application from day 1 to day 6 of 20 μl of the formulation to the right ear in Balb/c mice.

Evaluation method: Clinical observation and measurement of the thickness of the mouse ear from day 2 to day 12.

Weighing of the animals on day 1 and on day 12.

The placebo of composition 2 is not irritant.

### Example 7

### Tolerance Study

Study carried out on placebo formulas and composition 2 (containing 0.1% by weight relative to the total weight of the composition of {4-[6-ethyl-4′-(1-ethyl-1-hydroxypropyl)-2′-propylbiphenyl-3-yloxymethyl]-2-hydroxymethylphenyl}methanol).

After daily topical application, once a day for 6 days, of 20 μl of the formulation to the right ear in Balb/c mice.

Composition 2 induces the same response profile with an amplitude approximately 30% less than that of {4-[6-ethyl-4′-(1-ethyl-1-hydroxypropyl)-2′-propylbiphenyl-3-yloxymethyl]-2-hydroxymethylphenyl}methanol and 0.1% in ethanol; it does not induce any blood-calcium-grazing effect nor any weight loss.

The placebo of this formula does not induce an inflammatory response.

### Example 8

### Release/Penetration Study

Goal: To compare the in vitro percutaneous absorption of radiolabelled {4-[6-ethyl-4′-(1-ethyl-1-hydroxypropyl)-2′-propylbiphenyl-3-yloxymethyl]-2-hydroxymethylphenyl}methanol through human skin at 0.1% (m/m), from composition 2 and {4-[6-ethyl-4′-(1-ethyl-1-hydroxypropyl)-2′-propylbiphenyl-3-yloxymethyl]-2-hydroxymethylphenyl}methanol formulated in a carrier known for its high tolerance (control).

While the control formula is taken as reference and gives 100% absorption of {4-[6-ethyl-4′-(1-ethyl-1-hydroxypropyl)-2′-propylbiphenyl-3-yloxymethyl]-2-hydroxymethylphenyl}methanol, composition 2 gives 224% absorption of {4-[6-ethyl-4′-(1-ethyl-1-hydroxypropyl)-2′-propylbiphenyl-3-yloxymethyl]-2-hydroxymethylphenyl}methanol.

The compositions according to the invention thus allow twice as much penetration of the active agent.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. An anhydrous pharmaceutical composition, which comprises an organopolysiloxane elastomer and, as bioactive ingredient therein, at least one vitamin D compound, in a solubilized form, said vitamin D compound corresponding to general formula (I) below: 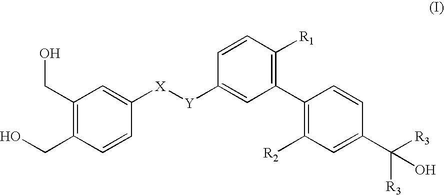
in which:
—X—Y— represents a link selected from among the following structures:
CH₂—CH₂—
CH₂—O—
O—CH₂—
—CH₂—N(R₄)—
with R₄ having the definition below,
R₁ is a methyl radical or an ethyl radical,
R₂ is an ethyl radical, a propyl radical or an isopropyl radical,
R₃ is an ethyl radical or a trifluoromethyl radical,
R₄ is a hydrogen atom, a methyl radical, an ethyl radical or a propyl radical.

2. The anhydrous pharmaceutical composition as defined by claim 1, said at least one vitamin D compound being selected from the group consisting of:
1. {5-[4′-(1-ethyl-1-hydroxypropyl)-6-methyl-2′-propylbiphenyl-3-yloxymethyl]-2-hydroxymethylphenyl}methanol;
2. {5-[6,2′-diethyl-4′-(1-ethyl-1-hydroxypropyl)biphenyl-3-yloxymethyl]-2-hydroxymethylphenyl}methanol;
3. {4-[6-ethyl-4′-(1-ethyl-1-hydroxypropyl)-2′-propylbiphenyl-3-yloxymethyl]-2-hydroxymethylphenyl}methanol;
4. {4-[6-ethyl-4′-(1-ethyl-1-hydroxypropyl)-2′-isopropylbiphenyl-3-yloxymethyl]-2-hydroxymethylphenyl}methanol;
5. (4-{2-[4′-(1-ethyl-1-hydroxypropyl)-6-methyl-2′-propylbiphenyl-3-yl]ethyl}-2-hydroxymethylphenyl)methanol;
6. {4-[4′-(1-ethyl-1-hydroxypropyl)-6-methyl-2′-propylbiphenyl-3-ylmethoxy]-2-hydroxymethylphenyl}methanol;
7. (4-{[4′-(1-ethyl-1-hydroxypropyl)-6-methyl-2′-propylbiphenyl-3-ylamino]methyl}-2-hydroxymethylphenyl)methanol;
8. [4-({[4′-(1-ethyl-1-hydroxypropyl)-6-methyl-2′-propylbiphenyl-3-yl]methylamino}methyl)-2-hydroxymethylphenyl]methanol;
9. [4-({ethyl-[4′-(1-ethyl-1-hydroxypropyl)-6-methyl-2′-propylbiphenyl-3-yl]amino}methyl)-2-hydroxymethylphenyl]methanol;
10. [4-({[4′-(1-ethyl-1-hydroxypropyl)-6-methyl-2′-propylbiphenyl-3-yl]propylamino}methyl)-2-hydroxymethylphenyl]methanol;
11. (2-hydroxymethyl-4-{2-[6-methyl-2′-propyl-4′-(2,2,2-trifluoro-1-hydroxy-1-trifluoromethylethyl)biphenyl-3-yl]ethyl}phenyl)methanol;
12. {2-hydroxymethyl-4-[6-methyl-2′-propyl-4′-(2,2,2-trifluoro-1-hydroxy-1-trifluoromethylethyl)biphenyl-3-yloxymethyl]phenyl}methanol;
13. {2-hydroxymethyl-4-[6-methyl-2′-propyl-4′-(2,2,2-trifluoro-1-hydroxy-1-trifluoromethylethyl)biphenyl-3-ylmethoxy]phenyl}methanol;
14. (2-hydroxymethyl-4-{[6-methyl-2′-propyl-4′-(2,2,2-trifluoro-1-hydroxy-1-trifluoromethylethyl)biphenyl-3-ylamino]methyl}phenyl)methanol;
15. [2-hydroxymethyl-4-({N-methyl-[6-methyl-2′-propyl-4′-(2,2,2-trifluoro-1-hydroxy-1-trifluoromethylethyl)biphenyl-3-yl]amino}methyl)phenyl]methanol;
16. [4-({N-ethyl-[6-methyl-2′-propyl-4′-(2,2,2-trifluoro-1-hydroxy-1-trifluoromethylethyl)biphenyl-3-yl]amino}methyl)-2-hydroxymethylphenyl]methanol;
17. [2-hydroxymethyl-4-({[6-methyl-2′-propyl-4′-(2,2,2-trifluoro-1-hydroxy-1-trifluoromethylethyl)biphenyl-3-yl]N-propylamino}methyl)phenyl]methanol;
18. (4-{2-[6-ethyl-4′-(1-ethyl-1-hydroxypropyl)-2′-propylbiphenyl-3-yl]ethyl}-2-hydroxymethylphenyl)methanol;
19. {4-[6-ethyl-4′-(1-ethyl-1-hydroxypropyl)-2′-propylbiphenyl-3-ylmethoxy]-2-hydroxymethylphenyl}methanol;
20. (4-{[6-ethyl-4′-(1-ethyl-1-hydroxypropyl)-2′-propylbiphenyl-3-ylamino]methyl}-2-hydroxymethylphenyl)methanol;
21. [4-({[6-ethyl-4′-(1-ethyl-1-hydroxypropyl)-2′-propylbiphenyl-3-yl]methylamino}methyl)-2-hydroxymethylphenyl]methanol;
22. [4-({ethyl-[6-ethyl-4′-(1-ethyl-1-hydroxypropyl)-2′-propylbiphenyl-3-yl]amino}methyl)-2-hydroxymethylphenyl]methanol;
23. [4-({[6-ethyl-4′-(1-ethyl-1-hydroxypropyl)-2′-propylbiphenyl-3-yl]propylamino}methyl)-2-hydroxymethylphenyl]methanol;
24. (4-{2-[6-ethyl-2′-propyl-4′-(2,2,2-trifluoro-1-hydroxy-1-trifluoromethylethyl)biphenyl-3-yl]ethyl}-2-hydroxymethylphenyl)methanol;
25. {4-[6-ethyl-2′-propyl-4′-(2,2,2-trifluoro-1-hydroxy-1-trifluoromethylethyl)biphenyl-3-yloxymethyl]-2-hydroxymethylphenyl}methanol;
26. {4-[6-ethyl-2′-propyl-4′-(2,2,2-trifluoro-1-hydroxy-1-trifluoromethylethyl)biphenyl-3-ylmethoxy]-2-hydroxymethylphenyl}methanol;
27. (4-{[6-ethyl-2′-propyl-4′-(2,2,2-trifluoro-1-hydroxy-1-trifluoromethylethyl)biphenyl-3-ylamino]methyl}-2-hydroxymethylphenyl)methanol;
28. [4-({[6-ethyl-2′-propyl-4′-(2,2,2-trifluoro-1-hydroxy-1-trifluoromethylethyl)biphenyl-3-yl]methylamino}methyl)-2-hydroxymethylphenyl]methanol;
29. [4-({N-ethyl[6-ethyl-2′-propyl-4′-(2,2,2-trifluoro-1-hydroxy-1-trifluoromethylethyl)biphenyl-3-yl]amino}methyl)-2-hydroxymethylphenyl]methanol;
30. [4-({[6-ethyl-2′-propyl-4′-(2,2,2-trifluoro-1-hydroxy-1-trifluoromethylethyl)biphenyl-3-yl]-N-propylamino}methyl)-2-hydroxymethylphenyl]methanol;
31. (4-{[4′-(1-ethyl-1-hydroxypropyl)-6,2′-dimethylbiphenyl-3-ylamino]methyl}-2-hydroxymethylphenyl)methanol; and mixtures thereof.

3. The anhydrous pharmaceutical composition as defined by claim 1, said at least one vitamin D compound comprising {4-[6-ethyl-4′-(1-ethyl-1-hydroxypropyl)-2′-propylbiphenyl-3-yloxymethyl]-2-hydroxymethylphenyl}methanol.

4. The anhydrous pharmaceutical composition as defined by claim 1, wherein said organopolysiloxane elastomer is not an adhesive.

5. The anhydrous pharmaceutical composition as defined by claim 1, formulated for topical application.

6. The anhydrous pharmaceutical composition as defined by claim 1, formulated as a salve or of an ointment.

7. The anhydrous pharmaceutical composition as defined by claim 1, said at least one bioactive ingredient being solubilized in a solvent.

8. The anhydrous pharmaceutical composition as defined by claim 7, said solvent being selected from the group consisting of absolute ethanol, oleyl macrogol 6 glycerides, macrogol 15 hydroxystearate, PEG 40 hydrogenated castor oil, PEG 400, dimethyl isosorbide, PPG 15 stearyl ether, ethoxydiglycol, N-methyl-2-pyrrolidone, and mixtures thereof.

9. The anhydrous pharmaceutical composition as defined by claim 8, said solvent comprising a mixture of absolute ethanol with at least one cosolvent selected from among oleyl macrogol 6 glycerides, macrogol 15 hydroxystearate, PEG 40 hydrogenated castor oil, PEG 400, dimethyl isosorbide, PPG 15 stearyl ether, ethoxydiglycol and N-methyl-2-pyrrolidone.

10. The anhydrous pharmaceutical composition as defined by claim 1, said organopolysiloxane elastomer being formulated in at least one volatile silicone oil selected from among linear or cyclic polyorganosiloxane oils containing from 2 to 10 silicon atoms, and optionally comprising alkyl or alkoxy radicals containing from 1 to 22 carbon atoms.

11. The anhydrous pharmaceutical composition as defined by claim 1, further comprising a feel additive selected from the group consisting of isopropyl palmitate, isopropyl myristate, C₁₂-C₁₅ alkyl benzoate, and mixtures thereof.

12. The anhydrous pharmaceutical composition as defined by claim 1, wherein the amount of organopolysiloxane elastomer ranges from 70% to 95% by weight relative to the total weight of the composition.

13. The anhydrous pharmaceutical composition as defined by claim 12, wherein the amount of the at least one vitamin D compound in a solubilized form ranges from 0.00001% to 5% by weight relative to the total weight of the composition.

14. The anhydrous pharmaceutical composition as defined by claim 13, wherein the amount of the at least one vitamin D compound in a solubilized form ranges from 0.0001% to 3% by weight.

15. The anhydrous pharmaceutical composition as defined by claim 14, wherein the amount of the at least one vitamin D compound in a solubilized form ranges from 0.0003% to 1% by weight.

16. The anhydrous pharmaceutical composition as defined by claim 7, wherein the amount of solvent ranges from 1% to 25% by weight relative to the total weight of the composition.

17. The anhydrous pharmaceutical composition as defined by claim 1, further comprising an anti-oxidizing agent selected from the group consisting of butylhydroxytoluene (BHT), butylhydroxyanisole (BHA), DL-alpha-tocopherol and propyl gallate.

18. The anhydrous pharmaceutical composition as defined by claim 1, further comprising a lipophilic anti-irritant selected from the group consisting of DL-alpha-tocopherol acetate, tea tree oil, green tea extract and calendula extract.

19. A process for preparing an anhydrous pharmaceutical composition as defined by claim 1, which comprises the following steps:
a) preparing phase A, comprising the organopolysiloxane elastomer, optionally mixed with an additional silicone oil and/or a feel additive, to homogeneity;
b) preparing phase B, by mixing at least one vitamin D compound of general formula (I) with the solvent, to homogeneity;
c) incorporating phase B into phase A, and then homogenizing until a homogeneous formulation is obtained.

20. The process for preparing an anhydrous pharmaceutical composition as defined by claim 19, said solvent comprising ethanol.

21. The process for preparing an anhydrous pharmaceutical composition as defined by claim 20, which comprises, at the beginning of step b), mixing the various cosolvents in ethanol, before introducing the vitamin D compound active agent.

22. The process for preparing an anhydrous pharmaceutical composition as defined by claim 19, carried out under cold conditions.

23. A regime or regimen for treating a disorder/affliction of the skin, comprising administering to a subject in need of such treatment, a thus effective amount of an anhydrous pharmaceutical composition as defined by claim 1.

24. A regime or regimen for treating psoriasis, comprising topically applying onto the affected skin area of a subject in need of such treatment, a thus effective amount of an anhydrous pharmaceutical composition as defined by claim 1.
